(19) United States
(12) Patent Application Publication
(10) Pub. No.: US 2022/0223264 A1
(43) Pub. Date: Jul. 14, 2022

(54) INFORMATION PROCESSING SYSTEM, NON-TRANSITORY COMPUTER READABLE MEDIUM STORING PROGRAM, AND INFORMATION PROCESSING METHOD

(71) Applicant: FUJIFILM Business Innovation Corp., Tokyo (JP)

(72) Inventors: Tetsuya KOBAYASHI, Kanagawa (JP); Kengo TOKUCHI, Kanagawa (JP)

(73) Assignee: FUJIFILM Business Innovation Corp., Tokyo (JP)

(21) Appl. No.: 17/377,330

(22) Filed: Jul. 15, 2021

(30) Foreign Application Priority Data
Jan. 12, 2021 (JP) 2021-003075

(51) Int. Cl. G16H 40/20; G06Q 10/02; G06Q 30/02; G07C 9/27; G05B 15/02; A61L 2/24; A61L 2/10

(52) U.S. Cl. CPC G16H 40/20; G06Q 10/02; G06Q 30/0284; G07C 9/00571; G05B 15/02; A61L 2/24; A61L 2/10; G07C 9/27

(57) ABSTRACT

An information processing system includes a processor configured to acquire behavior information that is information regarding a behavior of a user in a place used by the user, the behavior being a behavior that influences a degree of infection of an infectious disease with which the user infects another user via the place.

### CROSS-REFERENCE TO RELATED APPLICATIONS

This application is based on and claims priority under 35 USC 119 from Japanese Patent Application No. 2021-003075 filed Jan. 12, 2021.

### BACKGROUND

### (i) Technical Field

The present invention relates to an information processing system, a non-transitory computer readable medium storing a program, and an information processing method.

### (ii) Related Art

JP2010-217999A discloses a process of determines whether or not an entering/leaving person is wearing a mask and writing data as time information that is appropriate for wearing the mask into an IC card possessed by the entering/leaving person when the entering/leaving person is wearing the mask.

### SUMMARY

A place is shared, and other users may use this place following a user who used the place first. In this case, in a case where the user who used the place first has an infectious disease, other users may get the infectious disease via this place.

The degree of infection of the infectious disease to other users is not uniform, and, in a case where a user who uses the place first keeps the place clean and uses the place, the degree of infection decreases, and, in a case where the place is used in an unsanitary state, the degree of infection increases.

Aspects of non-limiting embodiments of the present disclosure relate to an information processing system and a non-transitory computer readable medium storing a program enabling to know a user's behavior that influences the degree of infection of an infectious disease with which a user infects other users via a shared place.

Aspects of certain non-limiting embodiments of the present disclosure address the above advantages and/or other advantages not described above.

However, aspects of the non-limiting embodiments are not required to address the advantages described above, and aspects of the non-limiting embodiments of the present disclosure may not address advantages described above.

According to an aspect of the present disclosure, there is provided an information processing system including a processor configured to acquire behavior information that is information regarding a behavior of a user in a place used by the user, the behavior being a behavior that influences a degree of infection of an infectious disease with which the user infects another user via the place.

### BRIEF DESCRIPTION OF THE DRAWINGS

Exemplary embodiment(s) of the present invention will be described in detail based on the following figures, wherein:

FIG. 1 is a diagram schematically showing an overall configuration of an information processing system;

FIG. 2 is a diagram showing a booth as an example of a space;

FIG. 3 is a diagram showing the inside of the booth;

FIG. 4 is a diagram showing an example of a hardware configuration of a space management server;

FIG. 5 is a diagram showing an example of a hardware configuration of a user terminal;

FIG. 6 is a diagram showing an example of a display screen displayed on a user terminal of a user of the booth when the user reserves the booth;

FIG. 7 is a diagram showing another example of a display screen displayed on the user terminal;

FIG. 8 is a flowchart showing a flow of processes executed by a CPU provided in a space management server when a user enters the booth reserved by the user;

FIG. 9 is a flowchart showing a flow of processes executed by the CPU after the user enters the booth;

FIG. 10 is a flowchart showing a flow of processes executed by the CPU after the user leaves the booth;

FIG. 11 is a flowchart showing a flow of schedule adjustment for booth cleaning;

FIG. 12 is a flowchart showing a flow of processes executed by the CPU regarding a booth usage fee and the like;

FIG. 13 is a flowchart showing a flow of processes executed by the CPU when the booth is actually cleaned;

FIGS. 14A and 14B are diagrams showing specific examples of schedule adjustment; and

FIG. 15 is a diagram showing a change of a reservation frame.

### DETAILED DESCRIPTION

Hereinafter, exemplary embodiments of the present invention will be described with reference to the drawings.

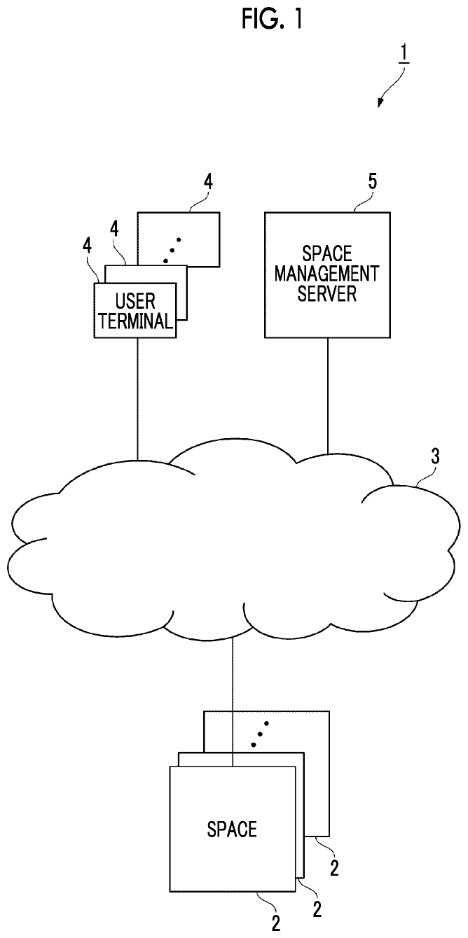

FIG. 1 is a diagram schematically showing the overall configuration of an information processing system 1 according to the present exemplary embodiment.

In the present exemplary embodiment, a plurality of spaces 2 that are examples of places reserved and used by users are provided.

In the present exemplary embodiment, each of the spaces 2 can be reserved, and the user reserves the space 2 in advance and then uses the space 2.

The space 2 as an example of a place includes a booth, a guest room such as an accommodation facility, and a conference room in a company and the like. These are examples of the space 2 separated from the surroundings by a wall, a partition, or the like.

The space 2 in the present exemplary embodiment includes a table, a seat, or the like for which a service is provided ata restaurant, a barber shop, or the like. These are examples of the space 2 open to the surroundings.

An information processing system 1 shown in FIG. 1 is configured with various terminals connected to a cloud network 3.

In FIG. 1, as an example of a terminal connected to the cloud network 3, a user terminal 4 operated by a user and a space management server 5 as an example of an information processing apparatus that manages the space 2 are shown. The space 2 is connected to the cloud network 3. More specifically, various apparatuses are provided in the space 2, and these apparatuses are connected to the cloud network 3.

In the present exemplary embodiment, an electronic lock is attached to a door of the space 2, and each of the spaces 2 can be locked. In the present exemplary embodiment, a person who has the authority to unlock the space 2 can use the space 2.

In a case of unlocking the space 2, the person performing unlocking operates the user terminal 4 thereof to give an instruction for the unlocking. This instruction is sent to the space management server 5, and the space management server 5 receives the instruction. The space management server 5 gives an instruction for unlocking the space 2 that the instruction for unlocking has been given. Consequently, the electronic lock installed in the space 2 is activated, and the space 2 is unlocked.

In the present exemplary embodiment, a portable smartphone is assumed as the user terminal 4. However, the portable user terminal 4 may be a so-called wearable terminal, a notebook computer, or a game terminal.

The space management server 5 manages various types of information related to the space 2. The space management server 5 manages, for example, information for specifying a user, information for specifying the space 2 that is a reservation target, the start date and time of the reservation, and the end date and time of the reservation.

The information for specifying a user includes, for example, the name, the gender, the age, an account, a user ID, a password of a user, and management information attached to an individual. The information for specifying the space 2 that is a use target includes, for example, information for specifying an address or a location, and a name or a number for management.

The space management server 5 also functions as a control apparatus and controls various apparatuses installed in the space 2.

The control apparatus may be installed in each space 2 so as to correspond to each of the spaces 2. In this case, the control apparatus installed in each space 2 controls various apparatuses installed in each space 2.

Appearance configuration of space 2

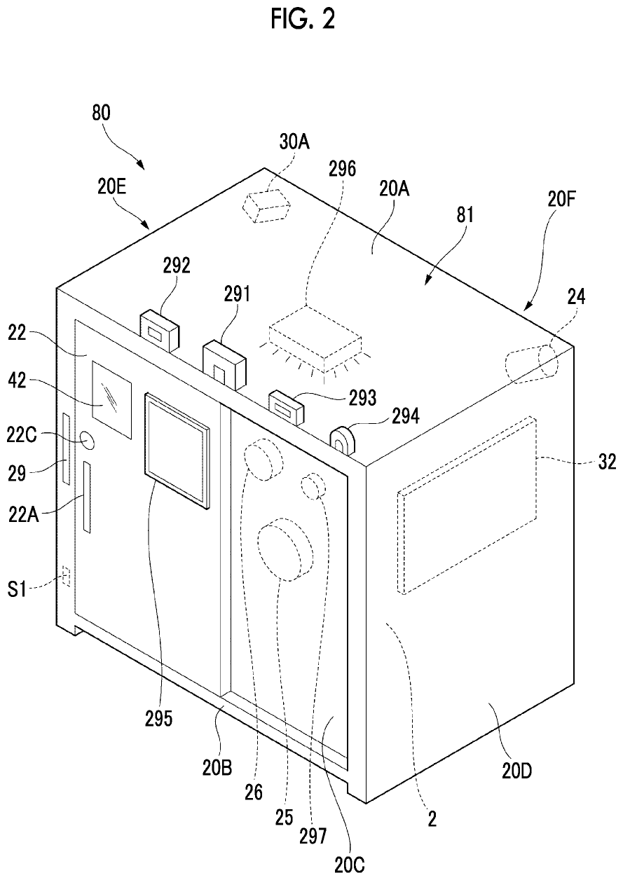

FIG. 2 is a diagram showing a booth 80 as an example of the space 2.

In the example shown in FIG. 2, the inside of the booth 80 is the space 2 that is a reservation target, and, in the present exemplary embodiment, the space 2 inside the booth 80 can be reserved.

The booth 80 of the present exemplary embodiment may be disposed indoors or outdoors, for example, in a station yard, an airport, an office building, a commercial facility such as a restaurant or a department store, a bank, a library, an art museum, a museum, a public institution or facility, a connecting passage, a park, etc.

The booth 80 shown in FIG. 2 is a closed type booth 80 to which a ceiling is attached.

Here, the “closed type” does not indicate a sealed type, but refers to a state having practical soundproofing performance.

The “booth 80” refers to a structure in which a partition for partitioning the space 2 and another space located around the space 2 is present. Here, partitions are not necessarily present on all four sides of the space 2, and even a structure in which partitions are not in some sides corresponds to the booth 80.

For example, in the space 2 used by a user while sitting, even a configuration in which there are two partitions only on the right side and the left side of the user corresponds to the booth 80.

The ceiling is not necessarily provided, and even a structure in which there is no ceiling corresponds to the booth 80.

The booth 80 shown in FIG. 2 is provided with a housing 81 that configures a part of the booth 80. The housing 81 is formed in a rectangular parallelepiped shape.

The booth 80 has a ceiling 20A, a floor surface 20B, a wall surface 20C to which a door 22 that can be opened and closed is attached, two wall surfaces 20D and 20E located on both sides of the wall surface 20C, and a wall surface 20F located on a side facing the door 22.

In the present exemplary embodiment, the space 2 is surrounded by the wall surface 20C, the door 22, the wall surface 20D, the wall surface 20E, and the wall surface 20F, and the space 2 is provided inside the four wall surfaces and the door 22.

In other words, in the present exemplary embodiment, the space 2 is provided inside the housing 81.

In the present exemplary embodiment, the door 22 is assumed to be a sliding door that is movable along the wall surface 20C. In the case of FIG. 2, the door 22 is a one-sided sliding door that slides in one direction, but may be a sliding door that is opened and closed by crossing two or more members, or a hinged door that slides two members left and right.

A handle 22A gripped by the user when opening and closing the door 22 is attached to the door 22. The handle is also provided inside the door 22.

An electronic lock 22C that enables unlocking and locking of the door 22 is attached to the door 22. In the present exemplary embodiment, an opening/closing sensor S1 for detecting opening/closing of the door 22 is provided.

The number of people using the booth 80 is substantially determined by a volume of the booth 80. The booth 80 in the present exemplary embodiment is basically assumed to be a private room type used by one person. However, the booth 80 may be a large booth 80 that can accommodate a large number of people.

The private room type does not indicate that only one person can use the booth, but indicates that a small number of persons, for example, two or three persons can use the booth.

Any shape or structure of the housing 81 configuring the booth 80 may be used, and any apparatus or performance thereof provided may be provided.

In the present exemplary embodiment, the ceiling 20A of the booth 80 is provided with an external imaging device 291 imaging an external situation of the booth 80 and an external microphone 292 acquiring external sound of the booth 80.

The external imaging device 291 is provided with an imaging element such as a CCD ora CMOS, and the external imaging device 291 uses the imaging element to image an external situation of the booth 80.

The ceiling 20A of the booth 80 is provided with a thermosensor 293 that detects a body temperature of a user outside the booth 80 and an external speaker 294 that is installed outside the booth 80 and outputs sound.

The booth 80 is provided with an internal microphone 297 that acquires sound inside the booth 80.

The door 22 of the booth 80 is provided with an external monitor 295 that displays information to users outside the booth 80.

A light source 296 that emits ultraviolet light, that is light including ultraviolet rays, is provided inside the booth 80, that is the ceiling 20A of the booth 80.

In the present exemplary embodiment, the inside of the booth 80 is cleaned by irradiating the inside of the booth 80 with ultraviolet light from the light source 296.

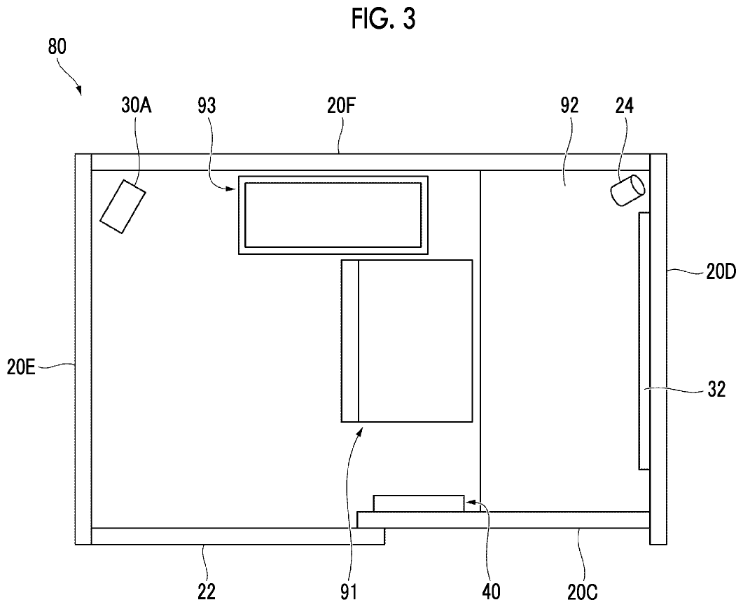

FIG. 3 is a diagram showing the inside of the booth 80. FIG. 3 shows a state in a case where the booth 80 is viewed from above.

In the present exemplary embodiment, a single desk 92 and a single chair 91 are disposed inside the booth 80.

In the booth 80, a luggage container 93 in which a user puts luggage thereof is installed. In addition, the luggage container 93 for accommodating the luggage placed by the user is provided in the booth 80.

As an installed apparatus, an internal monitor 32 displaying information is provided inside the booth 80 as shown in FIGS. 2 and 3.

In the present exemplary embodiment, as shown in FIGS. 2 and 3, an internal speaker 30A outputting sound is provided. The internal speaker 30A may not be provided separately, and the sound may be output from a speaker provided in the internal monitor 32.

In the present exemplary embodiment, as shown in FIGS. 2 and 3, an internal imaging device 24 imaging the inside of the booth 80 is provided. The internal imaging device 24 is provided with an imaging element such as a CCD or a CMOS, and the internal imaging device 24 uses the imaging element to image the inside of the booth 80.

As shown in FIG. 2, the booth 80 is provided with a human sensor 25 detecting a user inside the booth 80. In the present exemplary embodiment, a temperature sensor 26 detecting a temperature inside the booth 80 is provided.

As shown in FIG. 3, a lighting apparatus 40 brightening the inside of the booth 80 is provided in the booth 80.

In the present exemplary embodiment, as shown in FIG. 2, a window 42 is installed in the door 22, and, in the present exemplary embodiment, the inside of the space 2 can be visually recognized from the outside of the space 2 through the window 42.

As shown in FIG. 2, an information acquisition device 29 acquiring individual information of a user who uses the booth 80 may be provided on an outer surface of the booth 80.

The information acquisition device 29 is configured with, for example, a reader that reads a ID card that is put thereon. The information acquisition device 29 may be a reader or the like that reads a user's fingerprint, arrangement of veins, or the like.

Although not shown, the booth 80 is provided with an air conditioner adjusting a temperature inside the booth 80.

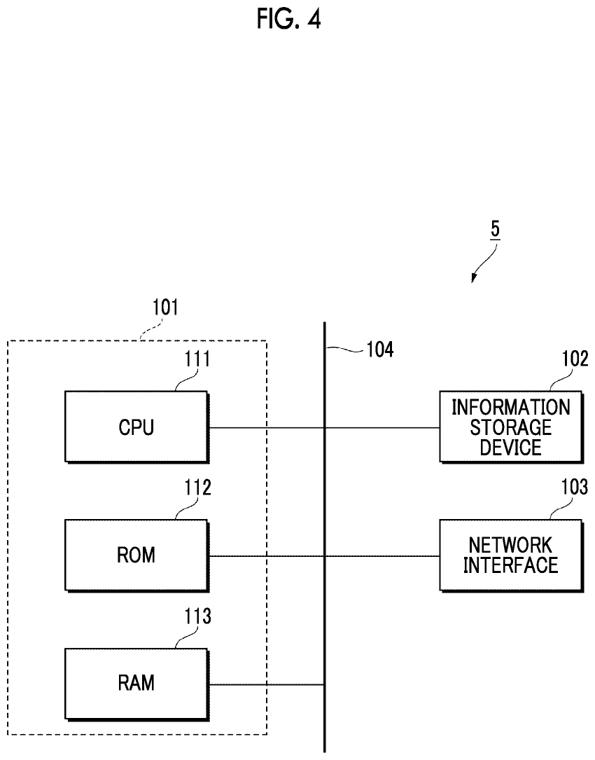

FIG. 4 is a diagram showing an example of the hardware configuration of the space management server 5.

The space management server 5 as an example of the information processing apparatus has a control unit 101 that controls an operation of the entire apparatus, an information storage device 102 that stores information, and a network interface 103 that realizes communication via a local area network (LAN) cable or the like.

The control unit 101 has a central processing unit (CPU) 111 as an example of a processor, a read only memory (ROM) 112 in which basic software, a basic input output system (BIOS), and the like are stored, and a random access memory (RAM) 113 that is used as a work area.

The CPU 111 may be multi-cores. The ROM 112 may be a rewritable non-volatile semiconductor memory. The control unit 101 is a so-called computer.

The information storage device 102 is configured with, for example, a hard disk drive. In other words, the information storage device 102 is configured with, for example, a device that reads and writes data to and from a non-volatile storage medium in which a magnetic material is coated on a surface of a disk-shaped substrate. However, the information storage device 102 may be a semiconductor memory or a magnetic tape.

The space management server 5 also includes input devices such as a keyboard and a mouse, and a display device such as a liquid crystal display as necessary.

The control unit 101, the information storage device 102, and the network interface 103 are connected to each other via a bus 104 or a signal line (not shown).

Here, a program executed by the CPU 111 may be provided to the space management server 5 in a state of being stored in a computer-readable recording medium such as a magnetic recording medium (a magnetic tape, a magnetic disk, or the like), an optical recording medium (an optical disk or the like), a magnetooptical recording medium, or a semiconductor memory.

The program executed by the CPU 111 may be provided to the space management server 5 by using communication means such as the Internet.

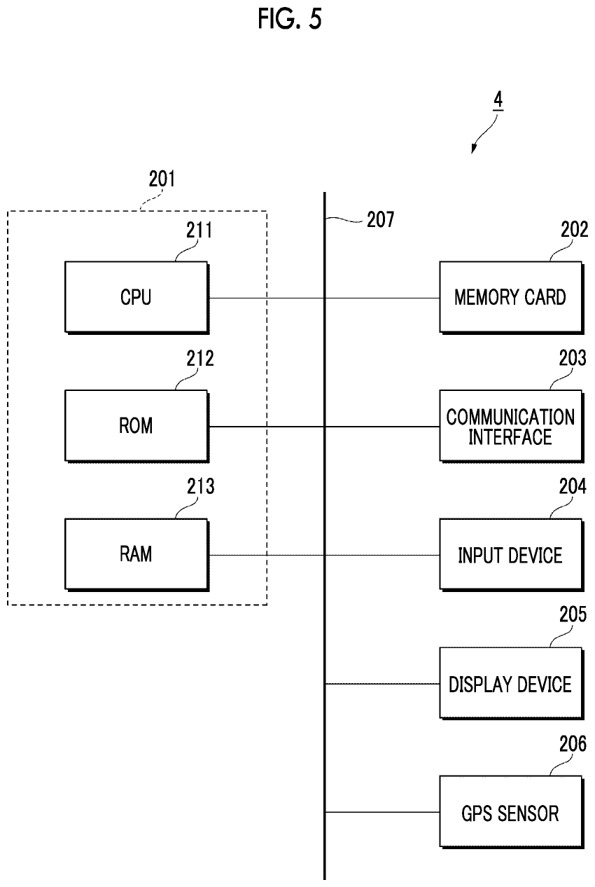

FIG. 5 is a diagram showing an example of a hardware configuration of the user terminal 4. In the configuration shown in FIG. 5, a case where the user terminal 4 is a smartphone is assumed.

The user terminal 4 has a control unit 201 that controls an operation of the entire apparatus, a memory card 202 that stores various data, various communication interfaces 203 that comply with wireless communication standards, an input device 204 such as a touch sensor, a display device 205 such as a liquid crystal display or an organic electroluminescence (EL) display, and a global positioning system (GPS) sensor 206.

The control unit 201 has a CPU 211, a ROM 212 in which firmware, a BIOS, and the like are stored, and a RAM 213 used as a work area. The CPU 211 may be multi-cores. The ROM 212 may be a rewritable non-volatile semiconductor memory.

The communication interface 203 is, for example, an interface used for connection to a mobile communication system or an interface used for connection to a wireless LAN.

The GPS sensor 206 is a sensor that receives radio waves from GPS satellites and measures a position of the user terminal 4. Latitude, longitude, and altitude information output from the GPS sensor 206 provides the current position of the user terminal 4. The GPS sensor 206 may be compatible with an indoor positioning system.

In the embodiments above, the term “processor” refers to hardware in a broad sense. Examples of the processor include general processors (e.g., CPU: Central Processing Unit) and dedicated processors (e.g., GPU: Graphics Processing Unit, ASIC: Application Specific Integrated Circuit, FPGA: Field Programmable Gate Array, and programmable logic device).

In the embodiments above, the term “processor” is broad enough to encompass one processor or plural processors in collaboration which are located physically apart from each other but may work cooperatively. The order of operations of the processor is not limited to one described in the embodiments above, and may be changed.

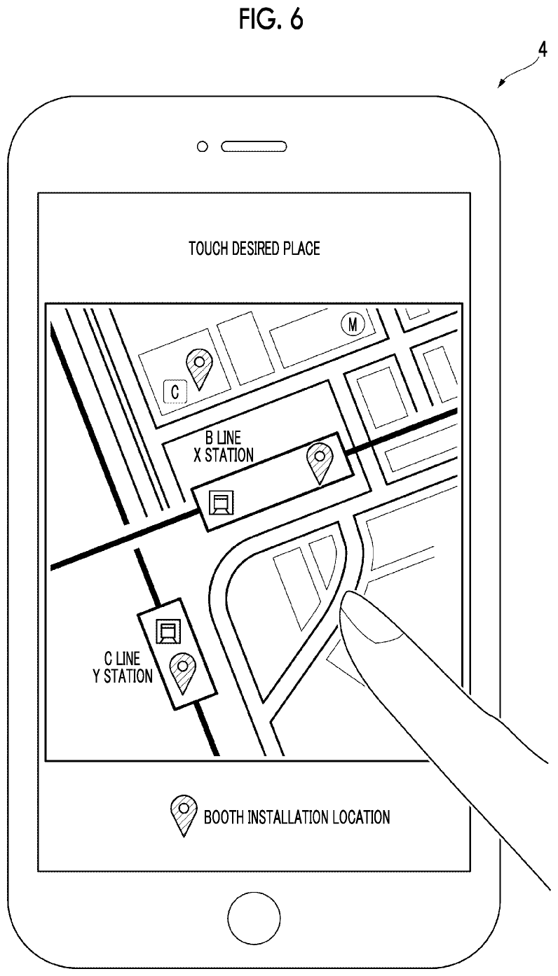

FIG. 6 is a diagram showing an example of a display screen displayed on the user terminal 4 of a user when the user of the booth 80 reserves the booth 80.

A map is displayed on the display screen shown in FIG. 6, and a plurality of installation locations of the booth 80 are displayed on the map.

In the present exemplary embodiment, when reserving the booth 80, first, the user of the booth 80 selects an installation location from among the plurality of displayed installation locations.

The present exemplary embodiment is not limited to such a display form, and for example, a plurality of installation locations may be displayed in a list form, and the user may select an installation location from the list.

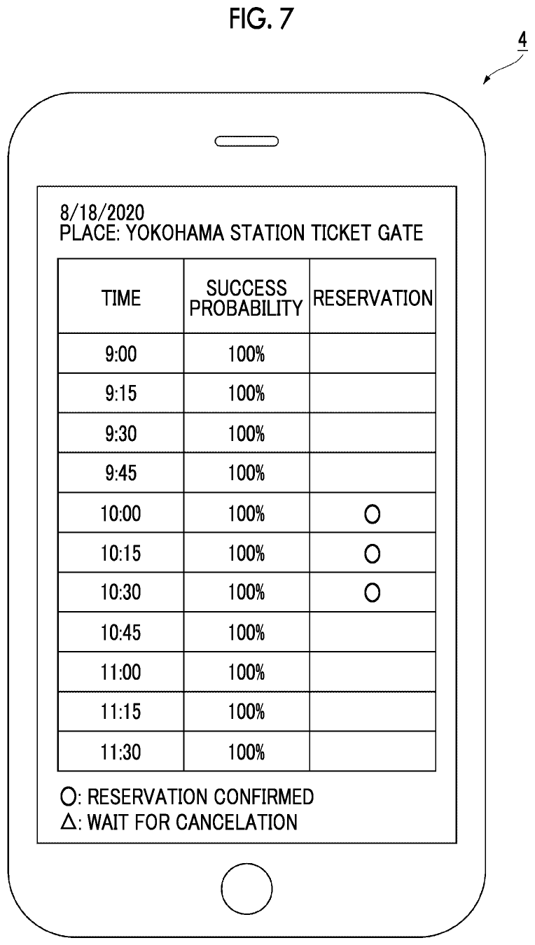

In a case where the installation location is selected, as shown in FIG. 7 (a diagram showing another example of the display screen displayed on the user terminal 4), a vacancy status at the selected installation location is displayed hourly on the user terminal 4.

The user performs an operation on the display screen to designate a reservation time for the booth 80. The user presses a confirmation button (not shown).

Consequently, the space management server 5 performs a reservation confirmation process.

Specifically, the space management server 5 receives information regarding the installation location of the booth 80 and the reservation time, and then registers the information regarding the installation location and the reservation time in the information storage device 102 (FIG. 4) to perform the reservation confirmation process. A confirmation result of the reservation is transmitted to the user terminal 4 and the reservation person is notified thereof.

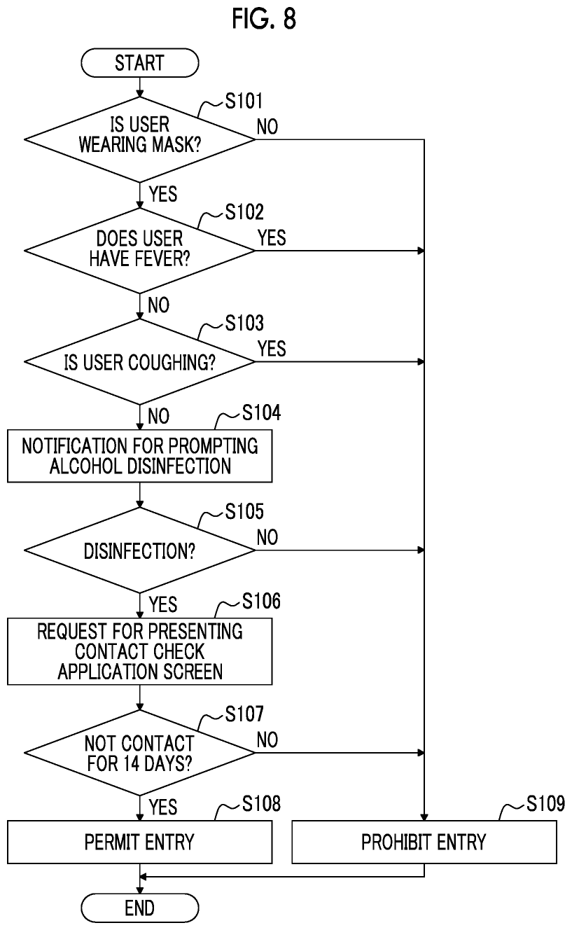

FIG. 8 is a flowchart showing a flow of processes executed by the CPU 111 provided in the space management server 5 when a user enters the reserved booth 80. Each process described below may be performed by a control apparatus (not shown) provided in the booth 80 instead of the space management server 5.

In the present exemplary embodiment, the CPU 111 acquires information regarding the user's status before the user enters the booth 80 that is an example of a place used by the user.

Specifically, first, when the user enters the booth 80, the CPU 111 determines whether or not the user is wearing a mask (step S101). Specifically, the CPU 111 analyzes an image obtained by the external imaging device 291 (refer to FIG. 2) to determine whether or not the user entering the booth 80 is wearing a mask.

In a case where the CPU 111 determines in step S101 that the user is wearing a mask, the CPU 111 determines whether or not the user has a fever based on an output from the thermosensor 293 (refer to FIG. 2) (step S102). In other words, the CPU 111 determines whether or not the user's fever exceeds a predefined threshold value.

In a case where the CPU 111 determines in step S102 that there is no fever, the CPU 111 determines whether or not the user is coughing (step S103). Specifically, the CPU 111 determines whether or not the user is coughing based on an image obtained by the external imaging device 291 or sound obtained by the external microphone 292 (refer to FIG. 2).

In a case where the CPU 111 determines in step S103 that the user is not coughing, the CPU 111 provides a notification for prompting the user to disinfect the fingers with alcohol (step S104).

Specifically, the notification for prompting the user to disinfect the fingers with alcohol is provided to the user outside the booth 80 via the external speaker 294 (refer to FIG. 2) or the external monitor 295.

Thereafter, the CPU 111 determines whether or not the user has performed alcohol disinfection based on an image acquired by the external imaging device 291 (step S105).

A storage container containing a disinfectant solution such as alcohol may be stored inside a storage section (not shown) provided with an electronic lock, for example. In this case, in a case where the user outside the booth 80 is a reservation person of the booth 80, the electronic lock may be unlocked such that this user can access the storage container containing the disinfectant solution.

Here, as to whether or not the user outside the booth 80 is a reservation person of the booth 80, for example, the user terminal 4 is asked to send identification information for authentication. Whether or not the user outside the booth 80 is a reservation person of the booth 80 is determined based on the identification information.

In a case where the CPU 111 determines in step S105 that the user has performed alcohol disinfection, the CPU 111 requests the user to present a screen of a contact check application (step S106).

The CPU 111 checks the screen and determines whether or not the user has contacted an infected person of an infectious disease in, for example, the past 14 days (step S107).

Here, the “infectious disease” refers to a disease in which a pathogen such as a bacterium or a virus invades the body and causes symptoms.

More specifically, in step S106 and step S107, first, the user's smartphone is directed toward the external imaging device 291 to acquire an image of the screen of the contact check application displayed on the smartphone.

The CPU 111 analyzes this image acquired by the external imaging device 291 to determine whether or not the user has contacted an infected person of an infectious disease in, for example, the past 14 days.

In a case where the CPU 111 determines that there is no contact between the user and an infected person, the CPU 111 performs determination to permit entry to the booth 80 (step S108).

Consequently, in the present exemplary embodiment, the booth 80 is unlocked such that the user can enter the booth 80.

The “contact check application” is an application that informs the user whether or not the user is near an infected person of an infectious disease.

In a case where the “contact check application” is installed in the user's smartphone, or the like, whether or not the user has been near the infected person is determined based on position information of the smartphone and position information of a smartphone possessed by the infected person described above.

In a case where the CPU 111 determines that the user is has been near the infected person, the user is notified of information indicating that the user has been near the infected person via the user's smartphone.

On the other hand, in a case where the CPU 111 determines that the user is not wearing a mask in step S101, the CPU 111 performs determination to prohibit the user from entering the booth 80 (step S109).

In other words, the CPU 111 performs determination to prohibit the user from entering the booth 80 in a case where a status of the user before entering the booth 80 is a specific status such as not wearing a mask.

Similarly, the CPU 111 performs determination to prohibit the user from entering the booth 80 in a case where the CPU 111 determines that the user has a fever in step S102 or the user is coughing in step S103 (step S109).

The CPU 111 also performs determination to prohibit the user from entering the booth 80 in a case where the CPU 111 determines that the user has not performed alcohol disinfection in step S105, or the user has contacted the infected person in step S107 (step S109).

In this case, the CPU 111 does not give an instruction for unlocking the booth 80, and the booth 80 remains locked.

In a case where the CPU 111 performs determination to prohibit the user from entering the booth 80, the CPU 111 notifies the user that the user cannot enter the booth 80. Specifically, the CPU 111 notifies the user that the user cannot enter the booth 80 via the user terminal 4, the external monitor 295, or the like.

In a case where the CPU 111 performs determination to prohibit the user from entering the booth 80, in a case where the user has already been charged, the CPU 111 performs a refund process.

In addition, in a charging form in which the user is charged after the user finishes using the booth 80, the CPU 111 does not charge the user in a case where a determination is made to prohibit the user from entering the booth 80.

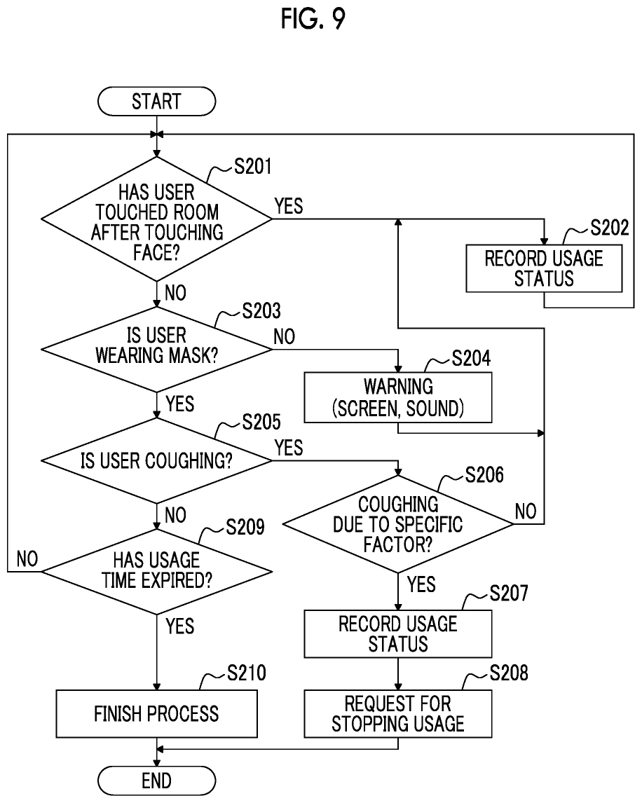

FIG. 9 is a flowchart showing a flow of processes executed by the CPU 111 after the user enters the booth 80.

After the user enters the booth 80, the CPU 111 performs processes in step S201 to step S210 shown in FIG. 9 every time a predefined time elapses. Specifically, the CPU 111 acquires behavior information that is information regarding behaviors performed by the user in the booth 80 every time a predefined time elapses.

More specifically, every time a predefined time elapses, the CPU 111 first analyzes the image obtained by the internal imaging device 24 to determine whether or not the user has touched the face thereof and then touches the room (the inside of the booth 80) (step S201).

In a case where the CPU 111 determines that the user has performed an action (behavior) of touching the room after touching the face, the CPU 111 records a usage status of the user (step S202). Specifically, in this case, the CPU 111 registers information indicating that the user has performed the action of touching the room after touching the face in the information storage device 102 (refer to FIG. 4).

On the other hand, in a case where the CPU 111 determines in step S201 that the user has not performed the action of touching the room after touching the face, the CPU 111 analyzes the image obtained by the internal imaging device 24 and determines whether the user is wearing a mask (step S203).

In a case where the CPU 111 determines that the user is not wearing the mask, the CPU 111 warns the user (S204). Specifically, the CPU 111 provides a notification for prompting the user to wear the mask via the internal monitor 32 or the internal speaker 30A.

Next, in this case, the CPU 111 performs the process in step S202 and records a usage status of the user. Specifically, in this case, the CPU 111 registers information indicating that the user is not wearing the mask in the information storage device 102.

In a case where the CPU 111 determines in step S203 that the user is wearing the mask, the CPU 111 determines whether or not the user is coughing (step S205).

Specifically, the CPU 111 determines whether or not the user is coughing in the booth 80 based on the image acquired by the internal imaging device 24 or the sound acquired by the internal microphone 297.

In a case where the CPU 111 determines that the user is coughing, the CPU 111 determines whether or not the cough is caused by a predefined specific factor such as a cough caused by a coronavirus (step S206).

In recent years, a technique has been developed in which sound of a cough obtained by a microphone is analyzed, and thus whether this cough is caused by a specific factor such as a coronavirus can be determined. In the process in step S206, the CPU 111 determines whether or not the cough is caused by a predefined specific factor by analyzing the sound of the cough.

In a case where the CPU 111 determines in step S206 that the cough is not caused by a specific factor, the CPU 111 performs the process in step S202 and records the usage status of the user. Specifically, in this case, the CPU 111 registers information indicating that the user has coughed in the information storage device 102.

Also in a case where the CPU 111 determines in step S206 that the cough is caused by a specific factor, the CPU 111 records the usage status of the user (step S207).

Specifically, in this case, the CPU 111 registers information indicating that the user has coughed due to a specific factor in the information storage device 102.

In this case, as shown in step S208, the CPU 111 requests the user in the booth 80 to stop using the booth 80 (step S208).

Specifically, the CPU 111 provides a notification for prompting the user to leave the booth 80 via, for example, the user terminal 4 or the internal monitor 32. Consequently, the user leaves the booth 80.

In other words, the CPU 111 provides a notification for prompting the user to leave the booth 80 in a case where the user has coughed due to a specific factor.

In other words, in a case where a behavior specified by the behavior information is a predefined specific behavior such as coughing caused by the coronavirus, the CPU 111 provides a notification for prompting the user to leave the booth 80.

Consequently, the user leaves the booth 80 without waiting for the end of the reservation time for the booth 80.

As described above, in a case where the user leaves the booth 80 without waiting for the end of the reservation time, in a case where the user has already charged, the charge for the unused time may be refunded.

Ina case of a charging form in which charging is performed after the booth 80 is used, the user may be charged for a time from the reservation start time for the booth 80 to a time when the user leaves the booth 80.

On the other hand, in a case where the CPU 111 determines in step S205 that the user is not coughing, the CPU 111 determines whether or not the usage time for the booth 80 has expired (step S209). In other words, the CPU 111 determines whether or not the reservation end time of the booth 80 has come.

In a case where the CPU 111 determines that the usage time for the booth 80 has expired, the CPU 111 finishes the process (step S210).

In the present exemplary embodiment, as described above, the CPU 111 acquires the behavior information that is information regarding the user's behavior in the booth 80 that is an example of a place used by the user.

More specifically, as described above, the CPU 111 acquires the behavior information that is information regarding a behavior that influences the degree of infection of an infectious disease with which the user infects other users via the booth 80, such as a behavior of touching the room, a behavior of wearing a mask, or a behavior of coughing.

Various behaviors such as the behavior of touching the room after the user touches the face, the behavior of the user wearing a mask, and the behavior of the user coughing all influence the degree to which other users using the booth 80 after the user are infected with the infectious disease.

In the present exemplary embodiment, information regarding the user's behavior that influences the degree of infection is acquired.

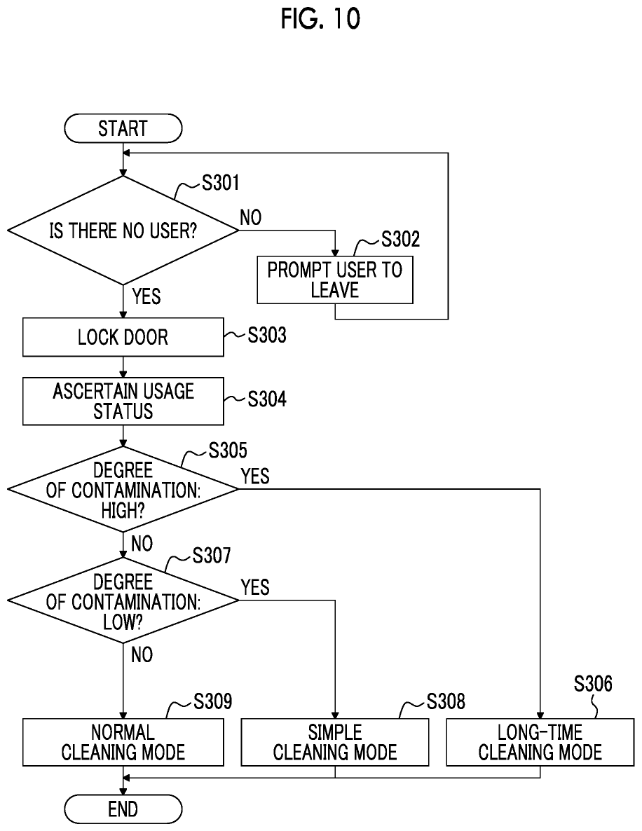

FIG. 10 is a flowchart showing a flow of processes executed by the CPU 111 after the usage time for the booth 80 expires.

In a case where the reservation end time (reservation end time point) has come, the CPU 111 determines whether or not there is no user in the booth 80 based on an output from the human sensor 25 (refer to FIG. 2) (step S301).

In a case where the CPU 111 determines that there is a user in the booth 80, the CPU 111 prompts the user in the booth 80 to leave the booth 80 via the internal speaker 30A or the internal monitor 32 (step S302).

On the other hand, in a case where the CPU 111 determines in step S301 that there is no user in the booth 80, the CPU 111 activates the electronic lock 22C (refer to FIG. 2) to lock the door 22 (step S303).

Next, the CPU 111 ascertains the usage status for the booth 80 by the user who has used the booth 80 before the locking (step S304).

Specifically, in the present exemplary embodiment, as described above, the usage status is registered in the information storage device 102, and, in step S304, the registered usage status is read from the information storage device 102 to ascertain the usage status of the user.

Next, the CPU 111 determines whether the degree of contamination of the booth 80 is high based on the ascertained usage status (step S305).

In a case where the CPU 111 determines that the degree of contamination of the booth 80 is high, the CPU 111 sets a cleaning mode of the booth 80 to a long-time cleaning mode (step S306).

In the present exemplary embodiment, in a case where the cleaning mode is set to the long-time cleaning mode, the inside of the booth 80 is irradiated with ultraviolet light from the light source 296 (refer to FIG. 2) during a first period of time.

In the present exemplary embodiment, a case where cleaning is performed by applying the ultraviolet light is described as an example, but the cleaning form is not limited to this, and a disinfectant solution maybe sprayed, a suction type robot vacuum cleaner may be operated, or a ventilation fan may be operated.

In the present exemplary embodiment, in a case where the degree of contamination of the booth 80 is determined as being high, the case where the cleaning mode of the booth 80 is set to the long-time cleaning mode is described as an example, but the present exemplary embodiment is not limited to this.

In a case where the degree of contamination of the booth 80 is determined as being high, an output during cleaning may be increased, for example, by increasing an output of the light source 296.

In cleaning the booth 80, the inside of the booth 80 is not limited to being cleaned uniformly, and cleaning may focus on a location of the booth 80 touched by a user or a location to which saliva (droplets) from the user's mouth is likely to be attached.

The location of the booth 80 touched by a user or the location to which saliva (droplets) from the user's mouth is likely to be attached may be ascertained by analyzing an image obtained by the internal imaging device 24.

The CPU 111 determines whether or not the degree of contamination of the booth 80 is high based on, for example, three conditions that the user's mask wearing time ratio exceeds 80%, the number of coughs is less than five, and the number of touching the room after the user touches the face is less than five.

In a case where any one of the three conditions is not satisfied, the CPU 111 determines that the degree of contamination of the booth 80 is high. In a case where all the three conditions are satisfied, the CPU 111 determines that the degree of contamination of the booth 80 is not high.

Here, the “mask wearing time ratio” refers to a ratio of the user's mask wearing time to the time for which the user has stayed in the booth 80.

In a case where the CPU 111 determines in step S305 that the degree of contamination of the booth 80 is not high, in step S307, the CPU 111 determines whether the degree of contamination of the booth 80 is low.

In a case where the CPU 111 determines that the degree of contamination of the booth 80 is low, the CPU 111 sets the cleaning mode of the booth 80 to a simple cleaning mode (step S308).

In a case where the cleaning mode is set to the simple cleaning mode, the inside of the booth 80 is irradiated with ultraviolet light during a short period of time. Specifically, the ultraviolet light is applied for a second period of time shorter than the first period of time described above.

In the present exemplary embodiment, in a case where the CPU 111 determines that the degree of contamination of the booth 80 is low, a case where the cleaning mode of the booth 80 is set to the short-time cleaning mode is described as an example, but a cleaning aspect is limited to this.

In a case where the degree of contamination of the booth 80 is determined as being low, an output during cleaning may be reduced by reducing an output of the light source 296.

The CPU 111 determines whether or not the degree of contamination of the booth 80 is low based on, for example, three conditions that the user's mask wearing time ratio exceeds 99%, the number of coughs is less than two, and the number of touching the room after the user touches the face is less than one.

The CPU 111 determines that the degree of contamination of the booth 80 is low in a case where all of the three conditions are satisfied, and determines that the degree of contamination of the booth 80 is not low in a case where at least one of the three conditions is not satisfied.

In a case where the CPU 111 determines in step S307 that the degree of contamination of the booth 80 is not low, that is, in a case where the degree of contamination of the booth 80 is neither high nor low and thus intermediate, the CPU 111 sets the cleaning mode of the booth 80 to a normal cleaning mode (step S309).

In a case where the normal cleaning mode is set, the ultraviolet light irradiation time is longer than that in the simple cleaning mode, and the ultraviolet light irradiation time is shorter than that in the long-time cleaning mode.

Specifically, in a case where the normal cleaning mode is set, ultraviolet light is applied for a third period of time that is shorter than the first period of time and longer than the second period of time.

In the present exemplary embodiment, as described above, the CPU 111 determines cleaning of the booth 80 based on the behavior information that is information regarding a behavior performed by the user in the booth 80 and influencing the degree of infection of an infectious disease.

Specifically, the CPU 111 determines a cleaning time for the booth 80 based on the behavior information.

More specifically, in the present exemplary embodiment, in a case where a behavior specified by the behavior information is a predefined specific behavior, the CPU 111 increases or reduces a cleaning time compared with a case where the behavior is not the specific behavior.

Specifically, for example, as described above, in a case where the behavior specified by the behavior information is the behavior of touching the room after the user touches the face, the CPU 111 increases the cleaning time.

On the other hand, for example, in a case where the behavior specified by the behavior information is not the behavior of touching the room after the user touches the face, the CPU 111 reduces the cleaning time. In other words, the CPU 111 reduces the cleaning time in a case where the user has performed the behavior of touching the room after the user touches the face thereof.

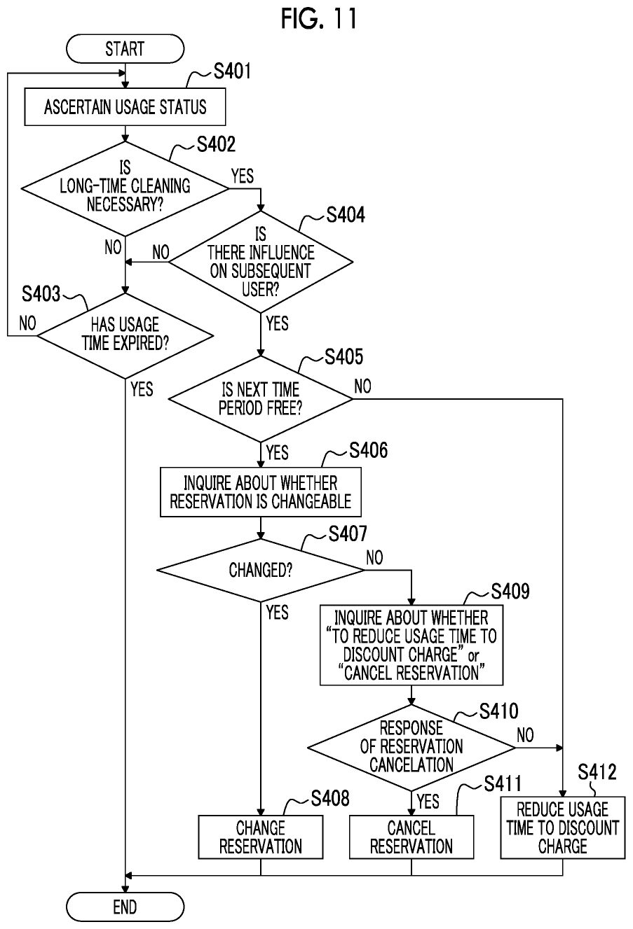

FIG. 11 is a flowchart showing a flow of schedule adjustment for cleaning the booth 80. In other words, FIG. 11 is a flowchart showing a flow of schedule adjustment for cleaning the booth 80 after the booth 80 has been used.

In schedule adjustment for cleaning, first, the CPU 111 ascertains a usage status for the booth 80 of a user (hereinafter, this user will be referred to as a “preceding user”) while the preceding user is using the booth 80 (step S401).

In the present exemplary embodiment, as described above, the usage status is registered in the information storage device 102 every time a predefined time elapses.

The CPU 111 reads the usage status from the information storage device 102 while the preceding user is using the booth 80, and ascertains the usage status of the preceding user.

The CPU 111 determines whether or not the booth 80 needs to be cleaned for a long time (hereinafter, referred to as “long-time cleaning” in some cases) based on the ascertained usage status (step S402).

Specifically, in this case, the CPU 111 determines whether or not the above three conditions are satisfied based on the ascertained usage status.

That is, the CPU 111 determines whether or not the three conditions that the mask wearing time ratio of the preceding user exceeds 80%, the number of coughs is less than five, and the number of touching the room after the preceding user touches the face is less than five are satisfied.

In a case where the CPU 111 determines in step S402 that the three conditions are satisfied and the booth 80 does not need to be cleaned for a long time, the CPU 111 determines whether or not the usage time for the booth 80 has expired (step S403).

In a case where the CPU 111 determines in step S403 that the usage time for the booth 80 has not expired, the CPU 111 returns to the process in step S401.

On the other hand, in a case where the CPU 111 determines in step S403 that the usage time for the booth 80 has expired, the CPU 111 finishes the process. In this case, the booth 80 is cleaned in the above-described normal cleaning mode.

On the other hand, in a case where the CPU 111 determines in step S402 that any of the three conditions is not satisfied and the booth 80 needs to be cleaned fora long time, the process proceeds to step S404.

In step S404, the CPU 111 determines whether or not the cleaning (long-time cleaning) performed after the usage time for the booth 80 expires influences a user who has made the next reservation (hereinafter, referred to as a “subsequent user”) (step S404).

In a case where the CPU 111 determines in step S404 that cleaning (long-time cleaning) does not influence the subsequent user, the CPU 111 performs the process in step S403 and determines whether or not the usage time (reservation time) has expired.

In step S403, in a case where the CPU 111 determines that the usage time has expired, the CPU 111 finishes the process.

In this case, long-time cleaning is performed after the usage time for booth 80 by the preceding user. In this case, this long-time cleaning does not influence the usage of the booth 80 by the subsequent user.

In contrast, in a case where the CPU 111 determines in step S404 that the long-time cleaning influences the subsequent user, the CPU 111 determines whether or not a time period next to the reservation time period reserved by the subsequent user is free (step S405).

In a case where the CPU 111 determines in step S405 that the next time period is free, the CPU 111 inquires the subsequent user about whether the reservation is changeable (step S406). Specifically, the CPU 111 inquires about whether the reservation is changeable via the user terminal 4 or the like. More specifically, the CPU 111 inquires about whether or not the reservation is changeable to the above next time period.

In a case where there is a response from the subsequent user, the CPU 111 determines whether or not to change the reservation based on this response (step S407).

In a case where the CPU 111 determines that the reservation is to be change, the CPU 111 changes the reservation (step 408). More specifically, the CPU 111 determines that the reservation is changed in a case where the subsequent user permits the change, and changes the reservation.

In other words, in this case, the CPU 111 changes the reservation of the subsequent user in a case where the subsequent user who is an example of another user different from the preceding user has given permission.

More specifically, in this case, the CPU 111 changes the reservation time of the reservation of the subsequent user.

In this case, the CPU 111 changes the reservation time of the subsequent user to the above next time period, and further notifies the subsequent user that the reservation has been changed.

More specifically, in this case, the CPU 111 makes a change to shift the start of the original reservation time of the subsequent user backward, and also makes a change to shift the end of the reservation time backward. The CPU 111 notifies the subsequent user that the reservation has been changed.

In this case (in a case where the reservation is changed), the CPU 111 gives an instruction for starting the long-time cleaning in a case where the reservation time of the preceding user has expired.

In this case, the long-time cleaning is performed over both a time period in which the long-time cleaning is originally scheduled and a time period that is free due to the above reservation change.

On the other hand, in a case where the CPU 111 determines in step S407 that the reservation is not to be changed, the CPU 111 inquires the subsequent user about whether to reduce the usage time to discount the charge or cancel the reservation (step S409).

The CPU 111 determines whether or not there is a response that the reservation is canceled (step S410). In a case where the CPU 111 determines that there is a response that the reservation is canceled, the CPU 111 cancels the reservation for the booth 80 of the subsequent user (step S411).

In other words, the CPU 111 cancels the reservation for the booth 80 of the subsequent user in a case where the subsequent user has given a permission.

In a case where a cleaning time of the cleaning performed after the use of the preceding user overlaps the reservation time for the booth 80 of the subsequent user, the reservation for the booth 80 of the subsequent user may be canceled as described above.

In the present exemplary embodiment, the cleaning time is determined, such as the above-described normal cleaning mode, simple cleaning mode, and long-time cleaning mode.

In the present exemplary embodiment, in a case where the long-time cleaning mode is determined and thus the cleaning time is determined to be a long cleaning time, a situation may occur in which the determined cleaning time overlaps the reservation time for the booth 80 of the subsequent user. In this case, as described above, the reservation for the booth 80 of the subsequent user may be canceled.

In this case, in a case where the reservation time of the preceding user has expired, the CPU 111 gives an instruction for starting the long-time cleaning.

In this case, the long-time cleaning is performed over both a time period in which the long-time cleaning is originally scheduled and a time period that is free due to the above reservation cancelation.

On the other hand, in a case where the CPU 111 determines in step S410 that there is no response that the reservation is canceled, the CPU 111 performs a process of reducing the usage time for the booth 80 of the subsequent user and discounting the charge (step S412).

Specifically, the CPU 111 performs setting to delay the reservation start time of the subsequent user and setting to discount the charge by an amount of the delayed time.

In other words, in this case, the CPU 111 makes a change to shift the start of the reservation time of the subsequent user backward, and performs setting to discount the charge by a time of shifting the start backward.

In this case, the CPU 111 does not shift the end of the reservation time backward.

In the present exemplary embodiment, as described above, in a case where the CPU 111 sets the cleaning mode to the long-time cleaning mode and thus the cleaning time is determined to be a long time, the cleaning time determined by the CPU 111 overlaps the reservation time for the booth 80 of the subsequent user.

In this case, as described above, a change to shift the start of the reservation time backward may be made. Consequently, the cleaning time and the usage time for the booth of the subsequent user do not overlap each other.

Also in this case, the CPU 111 gives an instruction for starting the long-time cleaning in a case where the reservation time of the preceding user has expired.

In this case, the long-time cleaning is performed over both a time period in which the long-time cleaning is originally scheduled and a time period that is free by shifting the start backward.

In the present exemplary embodiment, even in a case where the CPU 111 determines in step S405 that the next time period is not free, the CPU 111 executes the process in step S412 to perform a process of reducing the reservation time for the booth 80 of the subsequent user to discount the charge.

Also in this case, the long-time cleaning is performed over both a time period in which the long-time cleaning is originally scheduled and a time period that is free by shifting the start backward.

In a case where the CPU 111 determines in step S405 that the next time period is not free, the process in step S409 may be performed to inquire the subsequent user about whether to reduce the usage time to discount the charge or cancel the reservation.

In this case, in the same manner as above, the usage time for the booth 80 of the subsequent user is reduced and the charge is discounted, or the reservation for the booth 80 is cancelled.

In this case, in the same manner as above, the long-time cleaning is performed over both a time period in which the cleaning is originally scheduled and a time period that is free by shifting the start backward or canceling the reservation.

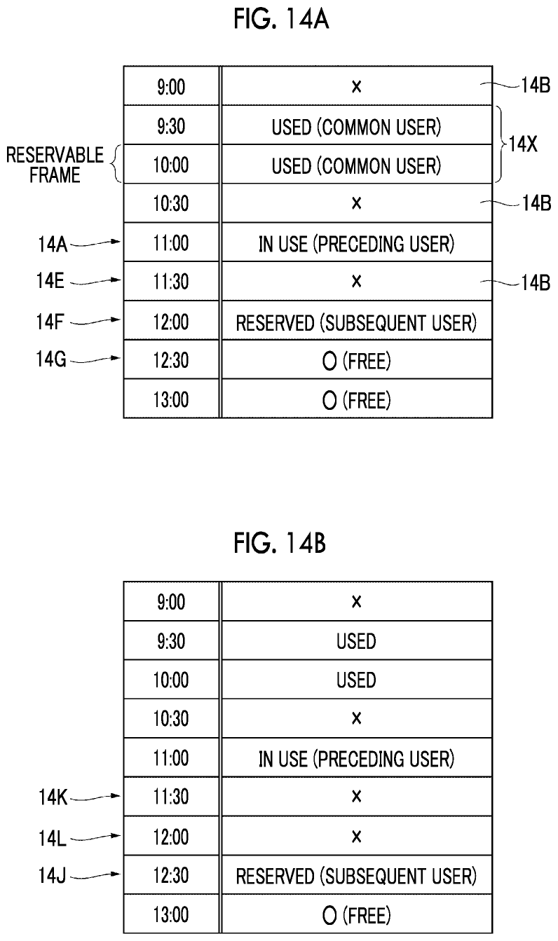

FIGS. 14A and 14B are diagrams showing specific examples of schedule adjustment.

In this example, a time period indicated by the reference sign 14A in FIG. 14A is a time period in which the preceding user is using the booth 80.

In this example, a process is performed in which reservations are not continuously made, and, thus, as indicated by the reference sign 14B, a free time period for which a reservation is not made is secured between reservation time periods that precede and succeed temporally.

In other words, in the present exemplary embodiment, a plurality of reservable frames are provided every 30 minutes, but a free time period for which a reservation is not made is secured between a reservable frame for which a reservation has already been made and the same reservable frame for which a reservation has already been made.

More specifically, in the present exemplary embodiment, in order to secure the cleaning time for the booth 80, a free time period for which a reservation is not made is secured between a certain reservable frame for which a reservation has already been made and another reservable frame for which a reservation has already been made.

In the present exemplary embodiment, the booth 80 is cleaned during the free time period. More specifically, in the present exemplary embodiment, cleaning in the normal cleaning mode, cleaning in the simple cleaning mode, and cleaning in the long-time cleaning mode are performed in this free time period.

Here, the “reservable frame” refers to a time frame of which the start and the end are predefined and in which a user reserves the booth 80.

In the present exemplary embodiment, as described above, the usage status of the preceding user is acquired. The long-time cleaning may be performed as described above depending on the usage status of the preceding user.

Here, in a case where the cleaning in the simple mode or the cleaning in the normal mode is performed, the cleaning is performed in a free time period indicated by the reference sign 14E in FIG. 14A.

In this case, since the cleaning is finished in this free time period, this cleaning does not influence a reservation time frame (reservation time period) reserved by the subsequent user, indicated by the reference sign 14F.

On the other hand, in a case where a status specified by the usage status of the preceding user is a status in which the degree of contamination is high, the long-time cleaning is performed.

In this case, cleaning is required to be performed not only in the free time period indicated by the reference sign 14E but also in the reservation time frame (time frame indicated by the reference sign 14F) reserved by the subsequent user, and thus this long-time cleaning influences the subsequent user.

In other words, in this case, the cleaning time of the long-time cleaning and the reservation time frame of the subsequent user temporally overlap each other, and thus the long-time cleaning influences the subsequent user.

In the present exemplary embodiment, in a case where the long-time cleaning is performed, whether or not the next reservation time frame of the reservation time frame of the subsequent user is free is determined as described above.

Specifically, in this example, whether or not a time frame indicated by the reference sign 14G in FIG. 14A is free is determined.

In this example, in a case where the time frame indicated by the reference sign 14G is free, the reservation is changed with the permission of the subsequent user, and the reservation time frame of the subsequent user is changed to the time frame indicated by the reference sign 14G in FIG. 14A.

FIG. 14B is a diagram showing a usage status and a reservation status of the booth 80 after the reservation time frame of the subsequent user is changed to the time frame indicated by the reference sign 14G in FIG. 14A.

In this example, as a result of changing the reservation time frame of the subsequent user, a new reservation time frame of the subsequent user is a time frame indicated by the reference sign 14J in FIG. 14B.

In this case, the long-time cleaning is performed over both a time frame indicated by the reference sign 14K in FIG. 14B (a time frame in which cleaning is scheduled from the beginning) and a time frame indicated by the reference sign 14L (a time frame that is free due to a reservation change).

On the other hand, for example, in a case where the time frame indicated by the reference sign 14G in FIG. 14A is not free, the reservation of the subsequent user cannot be changed.

In this case, as described above, the subsequent user is inquired about whether to reduce the usage time or cancel the reservation.

In a case where there is a response that the usage time is reduced, the long-time cleaning is performed over both a time frame indicated by the reference sign 14E in FIG. 14A (a time frame in which the cleaning is scheduled from the beginning) and a time frame that is free by reducing the usage time in the time frame indicated by the reference sign 14F.

In a case where there is a response that the reservation is canceled, the long-time cleaning is performed over both a time frame indicated by the reference sign 14K in FIG. 14B (a time frame in which the cleaning is scheduled from the beginning) and a time frame indicated by the reference sign 14L.

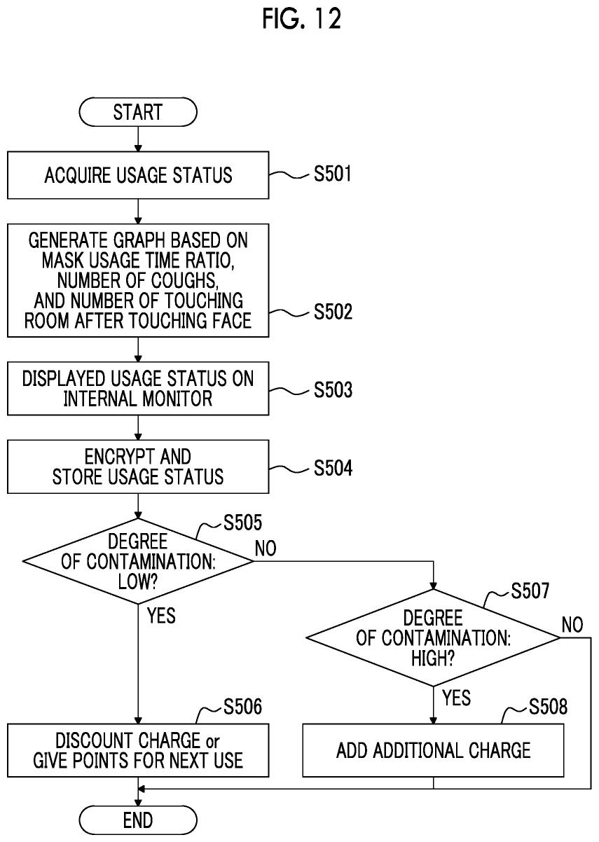

FIG. 12 is a flowchart showing a flow of processes executed by the CPU 111 with respect to a usage fee or the like for the booth 80.

Immediately before the reservation end time for the booth 80, the CPU 111 reads the usage status from the information storage device 102 and acquires the usage status in the same manner as described above (step S501).

The CPU 111 ascertains a mask usage time ratio, the number of coughs, and the number of touching the room after touching the face based on the acquired usage status, and generates a graph representing such information (step S502).

Next, the CPU 111 displays the usage status including the graph generated in step S502 on the internal monitor 32 (refer to FIG. 2) (step S503). As described above, displaying the usage status of the user and informing the user thereof raises the user's awareness of an infectious disease.

In a case where the user is informed of the usage status, a specific action among actions actually performed by the user in the booth 80 may be displayed on the internal monitor 32.

Specifically, for example, an image obtained by the internal imaging device 24, such as an image in which the user is not wearing a mask, an image in which the user is coughing, or an image in which the user touches the room after touching the face may be displayed on the internal monitor 32.

Thereafter, in the present exemplary embodiment, information regarding the usage status including the information included in the generated graph is encrypted and stored (step S504). Specifically, the usage status is encrypted and then registered in the information storage device 102.

Next, the CPU 111 determines whether or not the degree of contamination of the booth 80 is low based on the usage status acquired in step S501 (step S505).

Specifically, in the same manner as described above, the CPU 111 determines whether or not the degree of contamination of the booth 80 is low is based on, for example, three conditions that the user's mask wearing time ratio exceeds 99%, the number of coughs is less than two, and the number of touching the room after the user touches the face is less than one.

In a case where the CPU 111 determines in step S505 that the degree of contamination of the booth 80 is low, the CPU 111 discounts the charge for the booth 80 or gives points that can be allocated as a part of the charge when the booth 80 is used from the next time onward (step S506).

In the above description, the case of acquiring information regarding whether or not a user's behavior is a behavior of contaminating the booth 80 as the behavior information has been described, but acquired information is not limited to this, and information regarding a behavior of making the booth 80 clean, such as a behavior of cleaning the inside of the booth 80 may be acquired as the behavior information.

In a case where information regarding a behavior of cleaning the booth 80, such as a cleaning behavior, is acquired, the charge for the booth 80 may be discounted, or points that can be allocated as a part of the charge when the booth 80 is used from the next time onward may be given.

In a case of a pre-use charging form in which the charge for the booth 80 is discounted and charging is performed before using the booth 80, the discounted charge is refunded to the user.

In a case of a post-use charging form in which the charge for the booth 80 is discounted and charging is performed after the booth 80 is used, the discounted charge is added to the user.

On the other hand, in a case where the CPU 111 determines in step S505 that the degree of contamination of the booth 80 is not low, the CPU 111 determines whether the degree of contamination of the booth 80 is high (step S507).

Specifically, the CPU 111 performs a determination based on, for example, three conditions that the user's mask wearing time ratio does not exceeds 80%, the number of coughs is equal to or more than five, and the number of touching the room after the user touches the face is equal to or more than five.

In a case where any one of the three conditions is satisfied, the CPU 111 determines that the degree of contamination of the booth 80 is high. In this case, the CPU 111 adds an additional charge to the user (step S508).

Specifically, the CPU 111 further adds an additional charge in the case of the pre-use charging form, and adds, to the user, a charge obtained by adding an additional charge to the normal charge in the case of the post-use charging form.

On the other hand, in a case where the CPU 111 determines in step S507 that the degree of contamination of the booth 80 is not high, the process is finished without performing the process in step S508. In this case, the charge is the normal charge.

As described above, in the present exemplary embodiment, the CPU 111 determines the usage fee for the booth 80 based on the behavior information of the user.

Specifically, the CPU 111 performs determination to increase the usage fee in a case where a behavior specified by the behavior information is a behavior of increasing the degree of infection of an infectious disease.

On the other hand, the CPU 111 performs determination to reduce the usage fee in a case where a behavior specified by the behavior information is a behavior of reducing the degree of infection of an infectious disease.

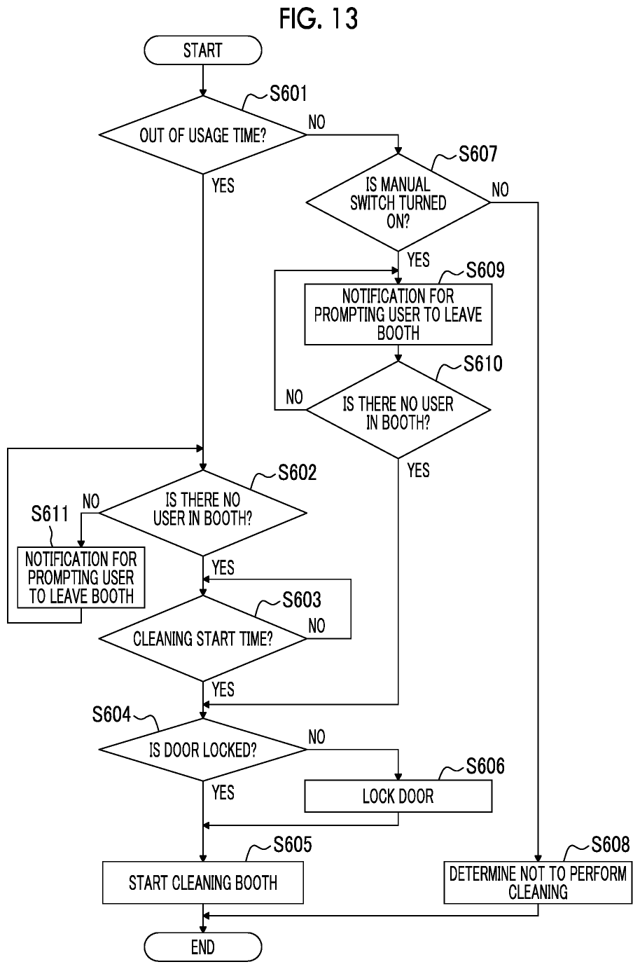

FIG. 13 is a flowchart showing a flow of processes executed by the CPU 111 in a case where the booth 80 is actually cleaned.

In the above description, the case where the booth 80 is cleaned after the usage time for the booth 80 is expired has been described, but the cleaning of the booth 80 may be performed during the usage time for the booth 80.

In this process shown in FIG. 13, a process of performing cleaning during the usage time for the booth 80 will also be described.

In actually cleaning the booth 80, the CPU 111 executes processes in steps 5601 to 5611 shown in FIG. 13 every time a predefined time elapses.

In cleaning the booth 80, first, the CPU 111 determines whether the booth 80 is out of the usage time (out of the reservation time) (step S601).

In a case where the CPU 111 determines that the booth 80 is out of the usage time, the CPU 111 determines whether or not there is no user in the booth 80 based on an output from the human sensor 25 (refer to FIG. 2) (step S602).

In a case where the CPU 111 determines that there is a user in the booth 80, the CPU 111 notifies the user in the booth 80 to leave the booth 80 via the internal speaker 30A or the internal monitor 32 (step S611).

On the other hand, in a case where the CPU 111 determines in step S602 that there is no user in the booth 80, the CPU 111 determines whether or not a predefined cleaning start time has come (step S603).

In a case where the CPU 111 determines that the cleaning start time has come, the CPU 111 determines whether the door 22 (refer to FIG. 2) is locked (step S604).

In a case where the CPU 111 determines that the door 22 is locked, the CPU 111 starts irradiation with ultraviolet light and starts cleaning the booth 80 (step S605).

On the other hand, in a case where the CPU 111 determines that the door is unlocked in step S604, the CPU 111 outputs a control signal for giving an instruction for locking the door 22 and locks the door 22 (step S606). Thereafter, the CPU 111 starts cleaning the booth 80 (step S605).

On the other hand, in a case where the CPU 111 determines in step S601 that the booth 80 is not out of the usage time, the CPU 111 determines whether or not a switch manually operated by the user (hereinafter referred to as a “manual switch”) is turned on (step S607).

An example of the manual switch includes a physical switch installed in the booth 80 and turned on/off by the user. In addition, an example of the manual switch includes a software switch displayed on the user terminal 4.

In a case where the CPU 111 determines in step S607 that the manual switch is not turned on, the CPU 111 determines not to perform cleaning (step S608).

On the other hand, in a case where the CPU 111 determines in step S607 that the manual switch is turned on, the CPU 111 provides a notification for prompting the user to leave the booth 80 via the internal monitor 32, the internal speaker 30A, or the like (step S609).

Next, the CPU 111 determines whether or not there is no user in the booth 80 (step S610). Specifically, the CPU 111 determines whether there is no user in the booth 80 based on an output from the human sensor 25 (refer to FIG. 2).

In a case where the CPU 111 determines that there is a user in the booth 80, the process in step S609 is performed again.

On the other hand, in a case where the CPU 111 determines in step S610 that there is no user in the booth 80, the CPU 111 executes the process in step S604.

In the process in step S604, the CPU 111 determines whether or not the door 22 is locked as described above. In a case where the CPU 111 determines that the booth 80 is locked, the cleaning of the booth 80 is started (step S605).

In this example, the cleaning started by turning on the manual switch is performed within the reservation time, and a user cannot use the booth 80 for the cleaning time.

As described above, in a case where the cleaning is performed within the reservation time, a fee for the cleaning time may not be charged.

Others

In the above description, basically, the case where the start and the end of the reservable frame is fixed has been described, but the reservable frame may be changed.

Specifically, in the above description, as shown in FIGS. 14A and 14B, for example, the case where the start of the reservable frame is 00 minutes past the hour or 30 minutes past the hour and the end of the reservable frame is also the same 00 minutes past the hour or 30 minutes past the hour has been described.

However, there is no limitation to this, and the start or the end of the reservation frame may be changed according to a usage status for the booth 80 of a preceding user. In other words, the start or the end of the reservation frame may be changed according to a status of cleaning performed after the booth 80 is used by the preceding user.

In the process described above, for example, the simple cleaning mode is set, and, even in a case where a cleaning time is less than 30 minutes, such as 5 minutes or 10 minutes, 30 minutes of free time for cleaning is secured. After 30 minutes elapses, the next reservable frame is started.

There is no limitation to, and the start of the next reservable frame after cleaning may be changed according to a length of the cleaning performed after the booth 80 is used by the preceding user.

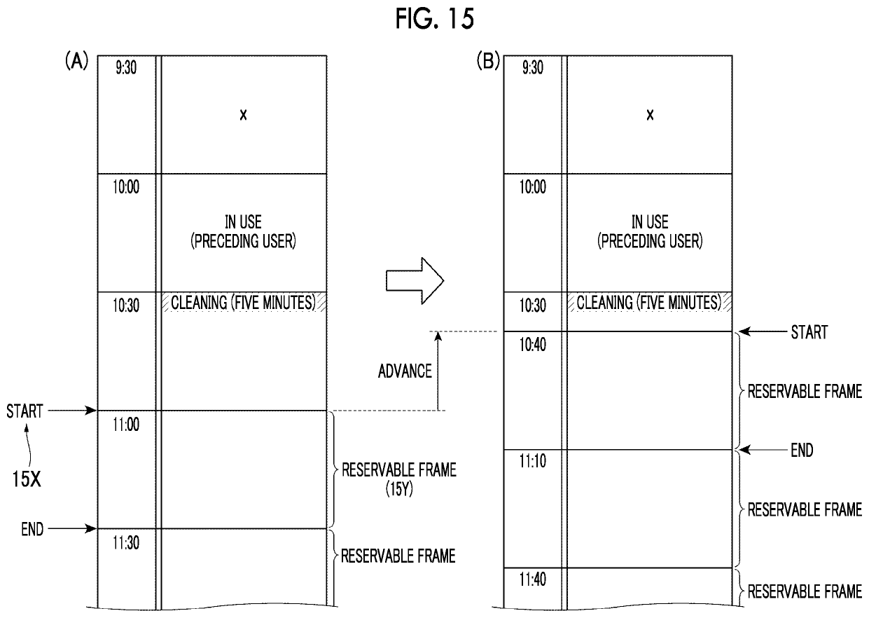

Here, for example, as shown in (A) of FIG. 15 (a diagram for describing a change of a reservation frame), a case is assumed in which, for example, cleaning for 5 minutes is confirmed after the use of the preceding user is finished. In other words, a case is assumed in which cleaning is determined to be performed in the simple cleaning mode, and, for example, cleaning is performed for 5 minutes after the use of the preceding user is finished.

In this case, the start (refer to the reference sign 15X) of a reservable frame (refer to the reference sign 15Y) present next to a time period in which the cleaning is performed may be advanced such that the reservable frame is started earlier as shown in (B) of FIG. 15.

In this case, the CPU 111 performs a process of advancing the start of the reservable frame that is set after the cleaning time. In other words, in this case, in a case where a time for cleaning performed after the use of the preceding user is reduced, the CPU 111 performs a process of advancing the start of the reservable frame that is set after the cleaning.

In this example, a process of advancing the end of the reservable frame is also performed.

In this example, the starts and the ends of other reservable frames after this reservable frame (the reservable frame indicated by the reference sign 15Y) are also advanced.

In a case where the process is performed, a situation in which the booth 80 becomes free and the booth 80 is not used even though the cleaning has been completed can be avoided.

In addition, a time period after a reservation time period (reservable frame) for which a reservation is made is not necessarily a free time period, and the free time period need not be secured depending on a reservation status.

In the present exemplary embodiment, as indicated by the reference sign 14B in FIG. 14A, a process in which reservations are not continuously made is performed, and thus a free time period for which a reservation is not made is secured between reservation time periods that precede and succeed temporally.

There is no limitation to this, this free time period need not be secured depending on a reservation status.

Specifically, for example, as indicated by the reference sign 14X in FIG. 14A, in a case where a plurality of consecutive reservation time periods are reserved by a common (same) user, free time period for the above cleaning need not be secured in the plurality of reservation time periods.

In a case where the booth 80 is used by a common user, infection between different users does not occur, and thus spread of an infectious disease can be suppressed without securing a cleaning time.

In other words, in a case where each of a plurality of temporally consecutive reservable frames is reserved by a common user, the CPU 111 permits this reservation. In this case, the above free time period is not secured, but the spread of an infectious disease can be suppressed without this free time period.

On the other hand, in a case where the plurality of temporally consecutive reservable frames are respectively reserved by different users, the CPU 111 does not permit a reservation for each of the plurality of temporally consecutive reservable frames.

In this case, after a certain user uses the booth 80, a free time is secured, and another user uses the booth 80 after the free time.

Each configuration described above is not limited to the above-described exemplary embodiment and the modification thereof and can be changed without departing from the spirit. In other words, it is understood that various changes in form and details are possible without departing from the purpose and scope of the claims.

For example, a part of each configuration described above may be omitted, or other functions may be added to each configuration described above.

Although a plurality of exemplary embodiments have been described above, the configuration included in one exemplary embodiment may be replaced with the configuration included in another exemplary embodiment, or the configuration included in one exemplary embodiment may be added to another exemplary embodiment.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An information processing system comprising:
a processor configured to acquire behavior information that is information regarding a behavior of a user in a place used by the user, the behavior being a behavior that influences a degree of infection of an infectious disease with which the user infects another user via the place.

2. The information processing system according to claim 1,
wherein the processor is configured to further perform determination on cleaning of the place based on the behavior information.

3. The information processing system according to claim 2,
wherein the processor is configured to perform determination on a cleaning time for the place based on the behavior information.

4. The information processing system according to claim 3,
wherein the processor is configured to, in a case where a behavior specified by the behavior information is a predefined specific behavior, increase or decrease the cleaning time compared with a case where the behavior is not the specific behavior.

5. The information processing system according to claim 3,
wherein the processor is configured to, in a case where the determined cleaning time overlaps a reservation time for the place of another user, change the reservation time for the place of the other user.

6. The information processing system according to claim 5,
wherein the processor is configured to, in a case where the reservation time is changed, make a change to shift a start of the reservation time backward.

7. The information processing system according to claim 6,
wherein the processor is configured to, in a case where the reservation time is changed, also make a change to shift an end of the reservation time backward.

8. The information processing system according to claim 5,
wherein the processor is configured to, in a case where there is permission of the other user, change the reservation time.

9. The information processing system according to claim 4,
wherein the processor is configured to, in a case where the cleaning time is decreased, advance a start of a reservable frame that is set after the cleaning time.

10. The information processing system according to claim 3,
wherein the processor is configured to, in a case where the determined cleaning time overlaps a reservation time for the place of another user, cancel a reservation for the place of the other user.

11. The information processing system according to claim 10,
wherein the processor is configured to, in a case where there is permission of the other user, cancel the reservation.

12. The information processing system according to claim 1,
wherein the processor is configured to, in a case where each of a plurality of temporally consecutive reservable frames is reserved by a common user, permit a reservation, and not to, in a case where the plurality of temporally consecutive reservable frames are respectively reserved by different users, permit reservations for the plurality of temporally consecutive reservable frames.

13. The information processing system according to claim 1,
wherein the processor is configured, in a case where a behavior specified by the behavior information is a predefined specific behavior, to provide a notification for prompting the user to leave the place.

14. The information processing system according to claim 1,
wherein the processor is configured to further perform determination on a usage fee for the place based on the behavior information.

15. The information processing system according to claim 14,
wherein the processor is configured, in a case where a behavior specified by the behavior information is a behavior of increasing the degree of infection of the infectious disease, to perform determination to increase the usage fee.

16. The information processing system according to claim 14,
wherein the processor is configured to, in a case where a behavior specified by the behavior information is a behavior of reducing the degree of infection of the infectious disease, perform determination to reduce the usage fee.

17. The information processing system according to claim 1,
wherein the processor is configured to further acquire information regarding a status of the user before the user enters the place used by the user.

18. The information processing system according to claim 17,
wherein the processor is configured to, in a case where the status of the user before entering the place is a specific status, perform determination to prohibit the user from entering the place.

19. A non-transitory computer readable medium storing a program causing a computer to realize a function of acquiring behavior information that is information regarding a behavior of a user in a place used by the user, the behavior being a behavior that influences a degree of infection of an infectious disease with which the user infects another user via the place.

20. An information processing method comprising:
acquiring behavior information that is information regarding a behavior of a user in a place used by the user, the behavior being a behavior that influences a degree of infection of an infectious disease with which the user infects another user via the place.
